(19) United States
(12) Patent Application Publication
(10) Pub. No.: US 2023/0404730 A1
(43) Pub. Date: Dec. 21, 2023

(54) CURING LIGHT AND THERAPEUTIC LASER SYSTEMS AND RELATED METHODS

(71) Applicant: CAO Group, Inc., West Jordan, UT (US)

(72) Inventors: Zhaohui Lin, Salt Lake City, UT (US); Densen Cao, Sandy, UT (US); Steven D. Jensen, South Jordan, UT (US)

(73) Assignee: CAO Group, Inc., West Jordan, UT (US)

(21) Appl. No.: 18/342,237

(22) Filed: Jun. 27, 2023

Related U.S. Application Data
(63) Continuation of application No. 18/298,275, filed on Apr. 10, 2023.
Application No. 18/298,275 is a continuation-in-part of application No. 17/631,491, filed on Jan. 30, 2022.
(60) Provisional application No. 62/879,926, filed on Jul. 29, 2019.

(51) Int. Cl. A61C 13/15

(52) U.S. Cl. CPC A61C 19/004

(57) ABSTRACT

By using techniques to diverge an emitted beam, the same base device may be utilized as either a cutting laser or a curing light. The use of multiple frequency laser modules (312) and a selection of emitter heads (304) allow for systems (300) which may be used for a wide variety of medical and dental procedures. Each system (300) has a plurality of emitter heads (304) to select from either a curing light, a cutting laser, or any other therapeutic energy emission all while utilizing the same laser module (312). Various structures of emitter heads are disclosed, as well as laser modules and connection strategies.

### CROSS-REFERENCES TO RELATED APPLICATIONS

This application claims priority as a continuation of U.S. application Ser. No. 18/298,275, filed Apr. 10, 2023, which is in turn a continuation-in-part of prior filed U.S. application Ser. No. 17/631,491, filed Jan. 30, 2022, which is in turn a § 371 national phase entry of PCT/US20/44039, filed Jul. 29, 2020, which claimed priority to prior filed U.S. Application No. 62/879,898, filed Jul. 29, 2019. These Applications are incorporated by reference herein in their entireties.

### FIELD OF THE INVENTION

The present invention relates to the field of medical, dental, and industrial instruments and more particularly relates to systems that may alternately emit radiant energy as either a therapeutic laser or a curing light or both.

### BACKGROUND OF THE INVENTION

In the past decades, humanity has harnessed the power of radiant energy for a multitude of purposes, particularly in the medical and dental fields. A curing light is an essential tool for use of light activated materials in a variety of industries. Particularly, curing lights are a daily tool that a practitioner uses in dentistry for curing composites, adhesives, and other materials. It is desirable for a curing light to have a high-power parallel beam with an adjustable beam size, no light degradation from light emitting positions between 10-20 mm, be compact with either corded or battery powered operation, and ideally be hand-held with an emitter head that fits within the confines of a typical oral cavity. Curing lights using LEDs as the light source are widely used in the industry today. The high intensity of LED curing lights has allowed curing times for conventional composites to be reduced from 40 seconds, as experienced with incandescent curing lights, to 10 seconds or less.

However, LEDs are dispersed light sources and not easily collimated within limited form factor. Therefore, a percentage of light emitted from LED light sources is wasted during the curing because the intensity of LED light reduces dramatically with distance. The ideal light source for curing is a collimated light source where light intensity does not change with distance.

Lasers have been used in various dental treatments since the 1900s. Therapeutic dental treatments where lasers have been used include treating tooth sensitivity, cavity detection and tooth preparation, surgery, pain management, healing, coagulation, and tooth whitening. Historically, a practitioner would need separate devices for different purposes (i.e., a laser for cutting or other therapeutic purposes and a curing light for material manipulation).

With the development of advanced diode lasers in different wavelength ranges, these diode lasers can serve as a source of radiant energy for a curing light. Commercially available diode lasers are of sufficient size and intensity that it provides improved options to manufacture laser-powered curing lights with significantly more light intensity output as compared to conventional LED curing lights. Dental composite can be cured in as little as 3 seconds, and sometimes only 1 second, of chair time under the appropriate operatory and clinical conditions. The size of laser diodes is similar to LED used in curing lights. Laser diodes are also efficient and can be powered by battery. As such they are more easily fit in a hand-held and battery-operated tool with other necessary components.

The present invention discloses using laser diode as a light source for curing light with uniformed beam profile and therapeutic devices for other purposes, combined or individually.

### SUMMARY OF THE INVENTION

In view of the foregoing disadvantages inherent in the known types of therapeutic lasers and curing lights, an improved device for curing or therapeutic or both functions may provide a system benefiting dental and medical practices. Such a device should meet the following objectives: that it provides effective laser and effective curing light functionality, that activation of either function be simple, intuitive, and efficient, that handheld and battery operated. As such, a new and improved radiant energy system may comprise a laser source combined with selectable emitter heads or tips which affect laser output from the laser source to accomplish these objectives. To create a uniformed beam, the device may utilize either a fiber with a divergent numerical aperture (“NA”) or a divergent lens or a lens system. The improved system may also utilize fiber optic cable to mix the light into a more uniform beam, particularly length of the fiber plays a role for better beam profile.

The more important features of the invention have thus been outlined in order that the more detailed description that follows may be better understood and in order that the present contribution to the art may be better appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions as far as they do not depart from the spirit and scope of the present invention.

### BRIEF DESCRIPTION OF THE DRAWINGS

FIG. 1 is a schematic drawing of a combination therapeutic laser and curing light system, utilizing a curing light head, as one embodiment of the invention.

FIG. 2 is the schematic of FIG. 1, where the combination therapeutic laser and curing light system is utilizing a cutting laser head.

FIG. 3 is the schematic of FIG. 1, where the combination therapeutic laser and curing light system is utilizing a diffuse laser head.

FIG. 4 is a schematic drawing of a combination therapeutic laser and curing light system, utilizing a curing light head, as a second embodiment of the invention.

FIG. 5 is the schematic of FIG. 4, where the combination therapeutic laser and curing light system is utilizing a cutting laser head.

FIG. 6 is the schematic of FIG. 4, where the combination therapeutic laser and curing light system is utilizing a diffuse laser head.

FIG. 7 is a schematic depicting one embodiment of a combination therapeutic laser and curing light system with a curing light head.

FIG. 8 is the combination therapeutic laser and curing light system of FIG. 7, with an alternate curing light attachment head.

FIG. 9 is the combination therapeutic laser and curing light system of FIG. 7, with another alternate curing light attachment head.

FIG. 10 is the combination therapeutic laser and curing light system of FIG. 7, with a further alternate curing light attachment head.

FIG. 11 is the combination therapeutic laser and curing light system of FIG. 7, with a still further alternate curing light attachment head.

FIG. 12 is the combination therapeutic laser and curing light system of FIG. 7, with an embodiment of a therapeutic cutting laser head.

FIG. 13 is the combination therapeutic laser and curing light system of FIG. 7, with an embodiment of a therapeutic large-area laser head.

FIG. 14 is a schematic drawing of a desktop system utilizing an embodiment of the combination therapeutic laser and curing light system.

FIG. 15 is a schematic drawing of a diode laser module for use in the combination therapeutic laser and curing light system.

FIG. 16 is a schematic drawing depicting an alternate diode laser module for use in the combination therapeutic laser and curing light system.

FIG. 17 is a schematic drawing depicting one embodiment of battery attachment for use in the combination therapeutic laser and curing light system.

FIG. 18 is a schematic drawing depicting one embodiment of head attachment for use in the combination therapeutic laser and curing light system.

FIG. 19 is a schematic drawing depicting optical properties of a fiber.

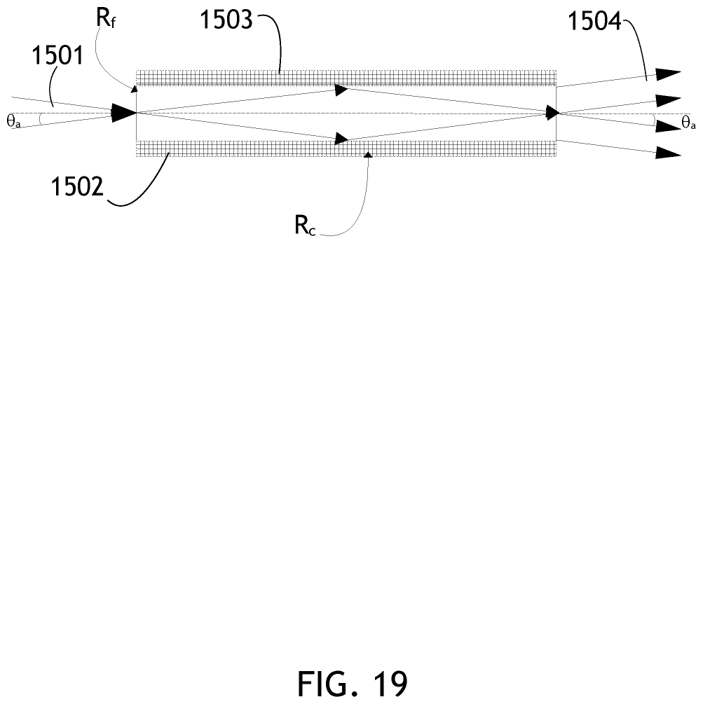

FIG. 20 is a schematic drawing depicting one embodiment of a therapeutic laser and curing light system utilizing a divergent lens.

FIG. 21 is a schematic drawing depicting three different types of lenses which may be utilized to diverge the beam.

FIG. 22 is a schematic drawing depicting one embodiment of a therapeutic laser and curing light system utilizing coiled fiber optic cable to diffuse light within the fiber.

### DETAILED DESCRIPTION OF THE PREFERRED EMBODIMENT

With reference now to the drawings, multiple embodiments of the combination therapeutic laser and curing light system are herein described. It should be noted that the articles “a,” “an,” and “the,” as used in this specification, include plural referents unless the content clearly dictates otherwise.

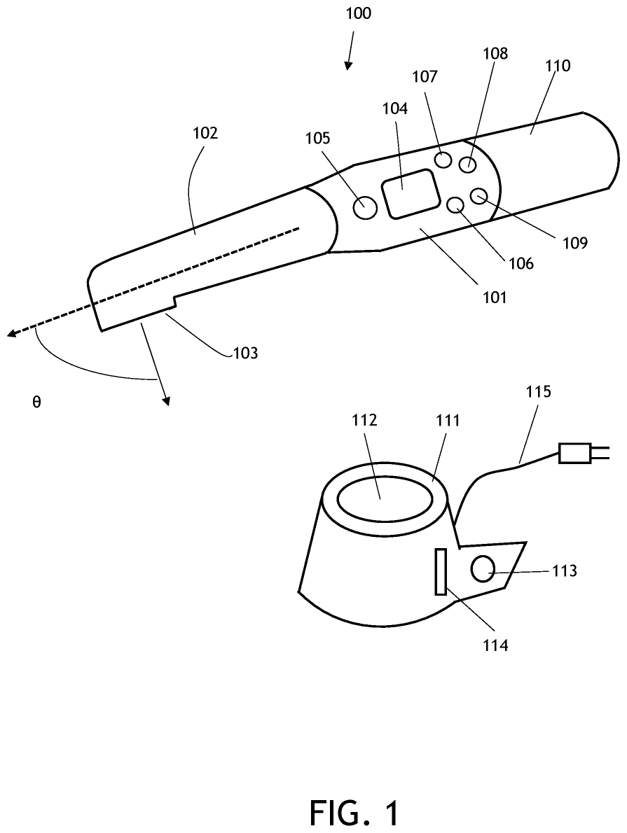

In FIG. 1, a curing light and laser system (100) features a main handpiece body (101) which can be made of metals, plastic, composite, or any other durable material. A curing light emitter head (102) includes a light exit (103). In some embodiments, the light exit (103) may be angled θ from 0 to 90 degrees in respect to a horizontal axis (dotted line in FIG. 1) of the curing head (102). The curing head (102) can also be made of metal, plastic, composite, or any other durable materials. The curing head (102) can be rotated around the main body (101) and is removable therefrom. A display (104) may show the light operation status. This display (104) can be LCD, OLED, LED module, or any other type of display. Various selection buttons may be provided for light activation (105), a timer (106) and to adjust operation modes (107), which could include the activation of one of a plurality of laser emitting chips on a laser module, each with a unique frequency. A main power on/off switch (108) and an emergency stop switch (109) may be utilized to stop light operation. The light can be powered by either AC/DC power or battery. If AC/DC power is used, the power source can plug into the light directly. Alternately a rechargeable battery (110) can be attached or detached from the main body (101). A charging station (111) may be provided for the battery (110) whereby a space (112) for either the battery (110) or the main body (101) may be provided. The battery (110) can be charged through contacts or wireless induction charging or may charge by plugging a cord into the unit. System status, in particular potential light output intensity, may be measured in the charging station (111) to have light placed on input window (113) and an intensity indicator (114) may be provided to show the intensity of light. The charging station (111) may be powered by a cord with an AC wall plug (115).

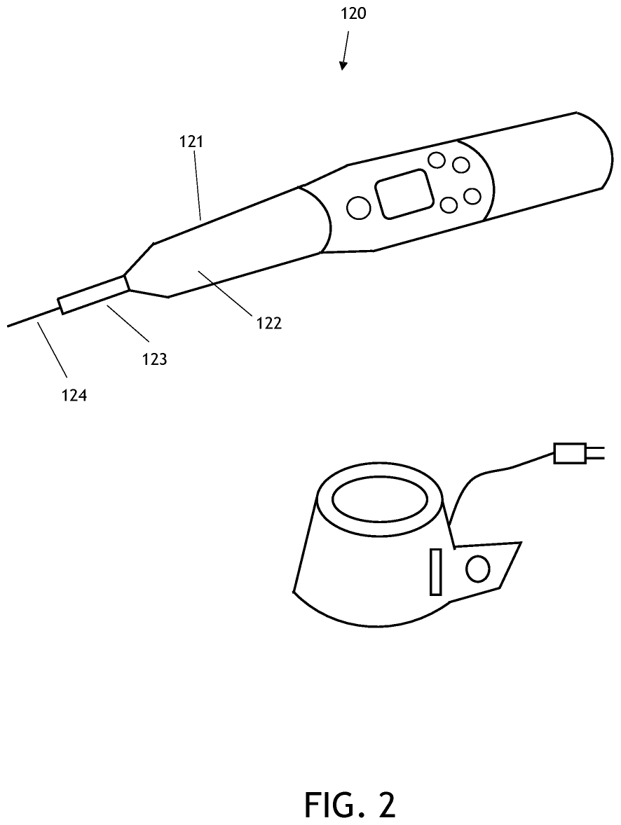

FIG. 2 shows a therapeutic laser system (120) which is achieved by changing the emitter head on the system described in FIG. 1. The embodiments shown are the same except the head of the light is changed to one utilizing a therapeutic cutting head which is useful for various therapeutic purposes. The main body (122) of the head (121) can be made of metal, plastic, composite, or any other durable material. While one end of the head (121) is attached to main body of the system, the other features a cannular portion (123) from which an optical fiber (124) is extruded. The cannular portion (123) can be made of metal or plastic and may be bendable to any angle as desired. The fiber (124) can have a size as small as 100 micrometers, but the entire head (121) may be configured to deliver different sizes or shapes of the beam.

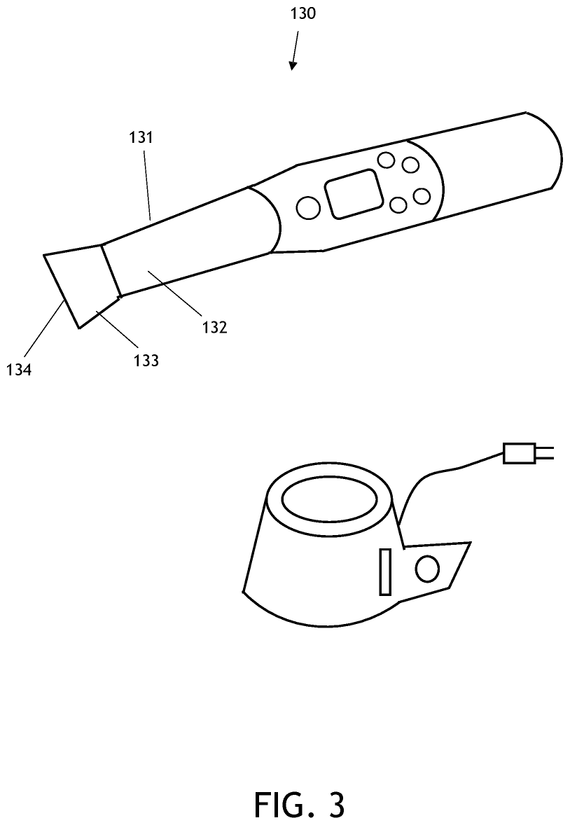

An alternate arrangement of the therapeutic system (130) may feature a head for therapy with a large area of beam (FIG. 3). Like the system (120) shown in FIG. 2, this therapeutic laser system (130) can also be achieved by changing the emitter head of the system described in FIGS. 1 and 2. All the embodiments are the same except that the head of light changed to a different therapeutic head (131). In this embodiment a head for other therapeutic purposes (131) features a main body (132) which can be made of metals, plastic, composite, or any other durable materials. Light exits (134) the head at a cone (133) and its size and shape may be altered by the size, shape of the cone (133), and optical system inside the head 131.

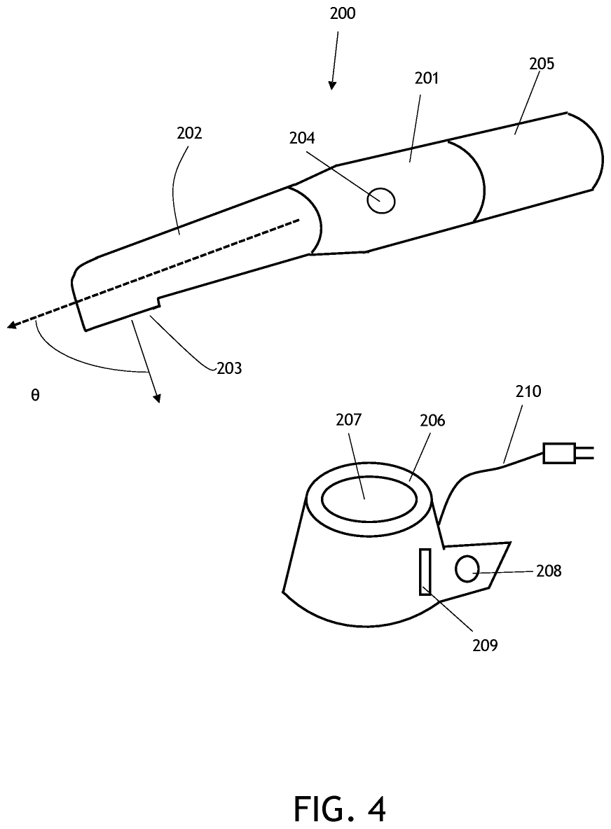

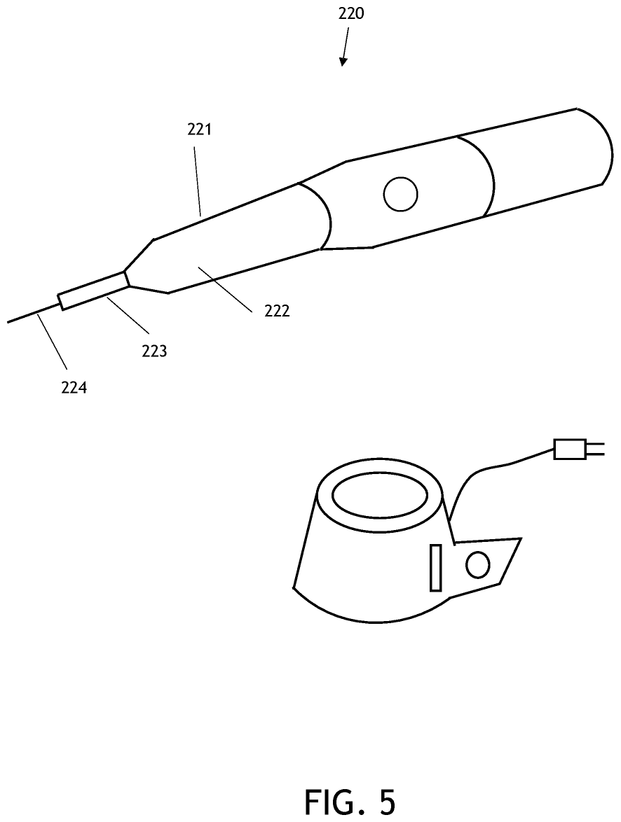

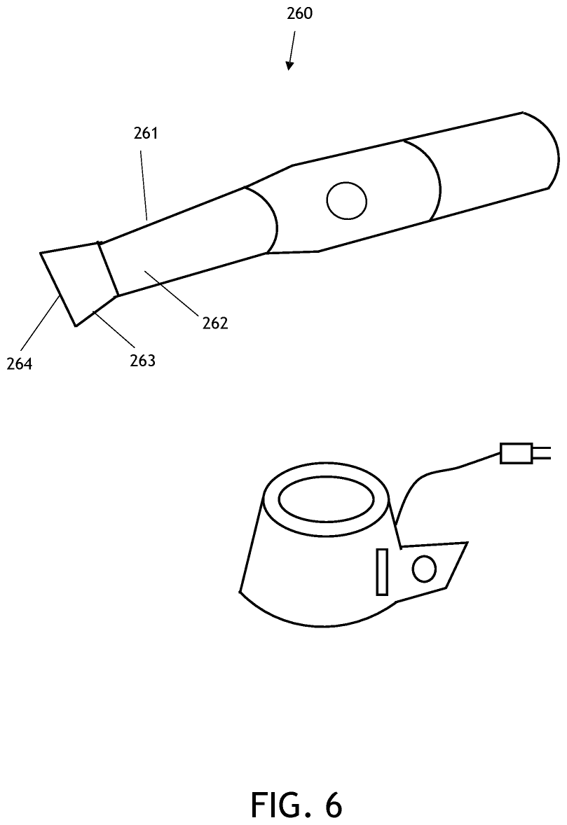

While the embodiments depicted in FIGS. 1-3 rely primarily on alternate heads to adapt the system to different purposes, a power control (107) is also provided to fine tune the system for given purposes. This control (107) is optional as the system can function for its purposes while relying entirely on the use of different heads. FIGS. 4-6 depict another schematic of invented system for curing light and therapeutic system using diode laser as light source, with a single switch. FIG. 4 is a curing light system and FIGS. 5 and 6 are therapeutic laser systems, respectively.

In FIG. 4, a curing light (200) is provided where (201) is the main body and with curing light emitter head (202) having a light exit (203). As with the previous embodiment, the direction of the exit (203) may be angled θ in a range 0 to 90 degrees respect to horizontal axis (dotted line in FIG. 2) of the curing head (202). As with the previous embodiment, the curing light (200) may be constructed of metal, plastic, composite, and any other durable materials and the curing head can be rotated around main body and be removable from the same. A single power button (204) may be provided with multiple functions. The button (204) can turn on and off the light for a fixed time, or cycle through frequency and power options. The button (204) may have multiple color backlights or separate indicator to indicate battery status and light emission status with different colors. The invented light can be powered by either AC/DC power or battery. If an AC/DC power is used, the power source can plug into the light directly and the power plug can be used as a main power switch and emergency stop. The unit may also be battery powered with a battery (205) that attaches the body (201) and that can be attached and detached easily to act as a main power switch and/or an emergency stop for the unit. A charging station (206) with an opening (207) for the battery or main body of the unit may also be provided. The charging station (206) is powered by a cord with wall plug (210). The battery (205) can be charged in a station with either contacts or wireless induction charging or by direct plugging in the unit to a power supply. As with the previous embodiment shown in FIGS. 1-3, system status may be measured and reported through a provided light intensity window (208) and an indicator (209).

A therapeutic cutting laser system (220) is achieved by changing the emitter head (221) of the system (FIG. 5). The therapeutic emitter head features a main body (222) can be made of metals, plastic, composite, and any other durable materials and features a cannular portion (223) through which an optical fiber (224) is extruded from the cannular portion. The cannular portion (223) can be made of metal or plastic and can be bendable to any angle as desired. The fiber (224) can be as small as 100 micrometers. As with the first system embodiment, the head (221) can utilize different configurations to deliver different sizes/shapes of the beam.

FIG. 6 depicts a large-beam therapeutic system (260). This embodiment is identical to the previous two, except that the head (261) emits a large beam for therapeutic purposes. As before, the main body (262) can be made of metals, plastic, composite, and any other durable materials. A cone (263) is located at the light exit (264) to allow passage of the broader beam. Beam size can be affected by the exit's size and shape and optical system inside the head (261).

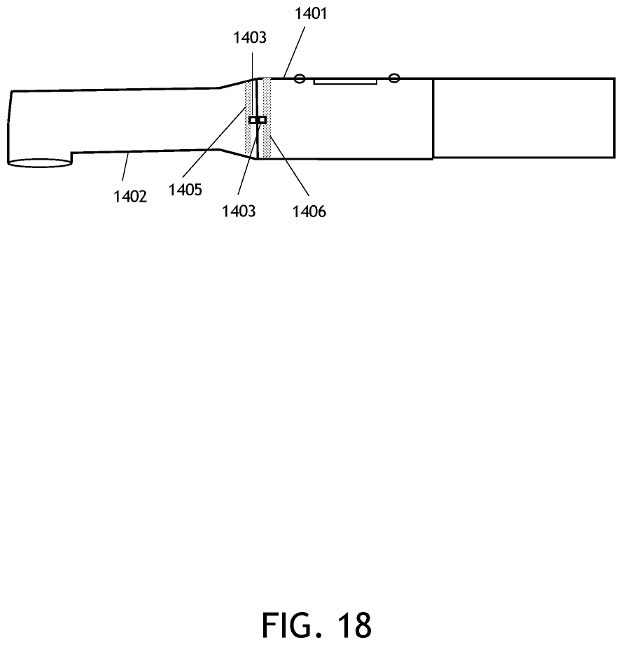

For the system to be used as both a laser and a curing light, the emitted beam from a diode laser needs to be diverged and collimated for use as curing light. An energy divergence system must be resident within the tool. The embodiments shown in FIGS. 1-17 illustrate using a fiber with a numerical aperture as component to collimate the beam. Numerical Aperture, or NA, is simply the angle at which light will enter and exit an optical system. It is based on the refractive indices of the media light is passing through. Generally, the higher the NA, the wider range of angles in which a system will receive, or emit, light. The basic concepts of numerical aperture are illustrated in FIG. 18, where light (1501) may enter a system so long as it hits the fiber (1502) within the angle θₐ from the center axis of the fiber. The light beam is then transmitted along the fiber, bouncing along the cladding walls (1503) until it exits the fiber (1504). In the context of a laser, θₐ is merely a measure of the divergence of an emitted beam. The refractive indices of the fiber R_{f} (1502) and the cladding R_{c} (1503) are usually different, and both are taken into account to determine the system's NA.

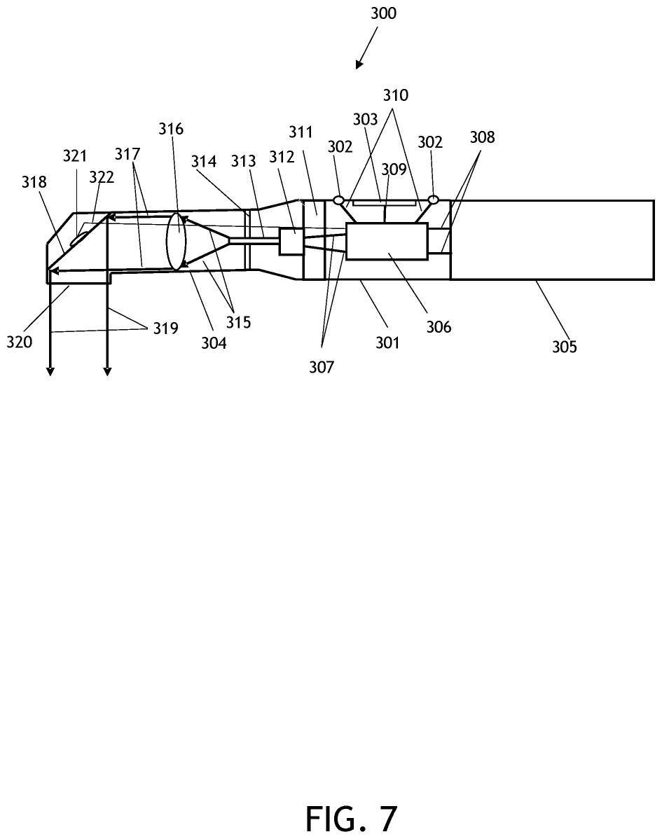

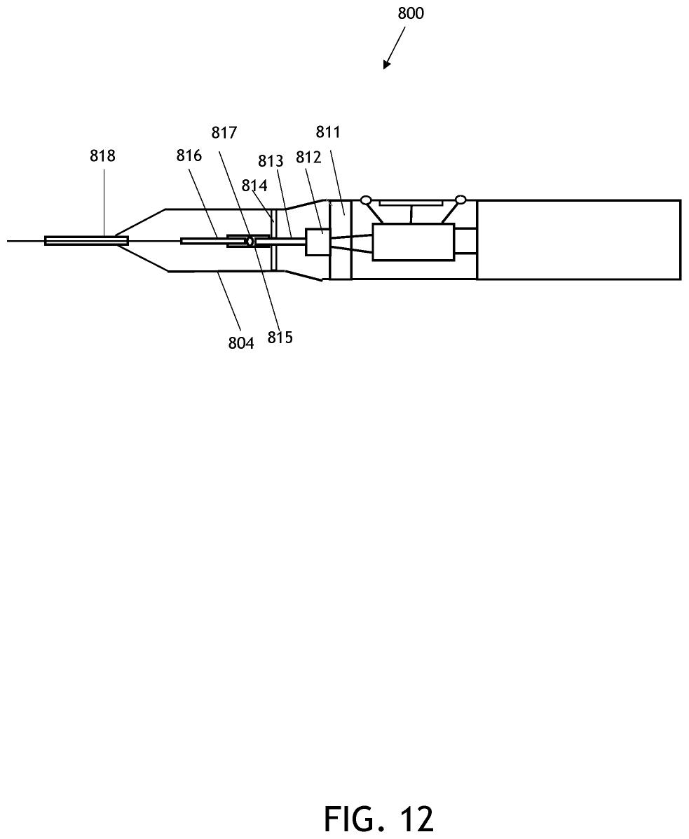

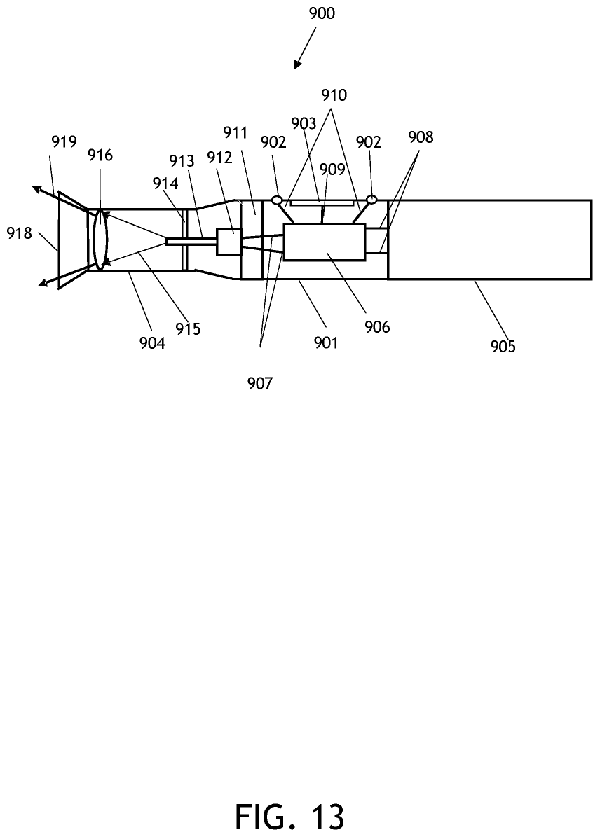

There are many potential designs for the device for different features and functions. FIGS. 7 through 11 depict various embodiments for curing light heads, while FIGS. 12 and 13 depict therapeutic heads. These designs are exemplary, and do not necessarily work with each other, but are shown to depict some of the many designs which may be utilized in the practice of this invention. In FIG. 7, one embodiment of the system handpiece (300) features a main handpiece housing (301) with control buttons (302) and a display (303). A head housing (304) is removable and exchangeable from handpiece body (301) while a battery or other power supply (305), such as an AC/DC power supply, is also provided. A control circuit (306) controls the light power output, laser operation control (including time), output power, pulse rate, battery status, and other features that are required for curing light and laser system operations. There are connections from control circuit to different components: (307) including connections (307) to laser module (312); connections (308) to a battery or AC/DC power supply (305); connection (209) to a display (303); and connections (310) to control button(s)(302). The laser module (312) is ideally mounted upon a heat sink (311). At this point, an optical system, which includes fiber (313), collimating lens (316) and reflector (318), converts the light emitted from laser module (312) into a collimated beam (319). In this embodiment, the collimated beam with a size can be ranged from 2 to 14 mm and the divergent angle of the beam θₐ is less than 10 degrees. The beam size can also be adjusted by attaching a fixture with different aperture size at beam exit point. The aperture size can be 2, 4, 6, and 8 mm. The fixture can also contact a filter to adjust beam intensity as needed. A fiber (313) attached to laser module (312) initially collects emitted light and directs beam (315) into collimating lens (316) which then converts the beam into a collimated, parallel beam (317), as is required in curing operations. Fiber (313) may be terminated with ferrule or be free standing with cleaved interface on the fiber side. The length of the fiber (313) depends on the requirement of head (304) and can be ranged from 1 mm to 1000 mm. The size or diameter of the fiber can be ranged from 50 to 1000 μm. A holder (314) may be used to hold the fiber (313) into a position. The laser module 312 can be placed in front of control circuit 306 or any position in or top or bottom of the circuit 306 or behind the circuit 306. The position of the lens from end of fiber depends on the focal length of the collimating lens (316) and the size of the parallel beam (317) will depend on diameter of the collimating lens (316). The parallel beam (317) travels to a reflector (318) which will turn the beam (317) as required for the geometry of the head (304). The depicted reflector (318) is positioned at a 45-degree angle in respect to lens (316) to turn the light beam to 90-degree direction to form a beam (319) to reach to wand exit (320). The position or angle of the reflector (318) can vary to conduct light in different directions and along different angles. The distance between lens and reflector depends on the requirement of head length. A photo detector (321) may be provided to measure the light intensity and feedback the signal through connection (322) to control circuit (306), which may then adjust the light intensity based on this feedback signal. All the components after the fiber holder (314) are in the head housing (304) and can be removed along with the head from handpiece body (301).

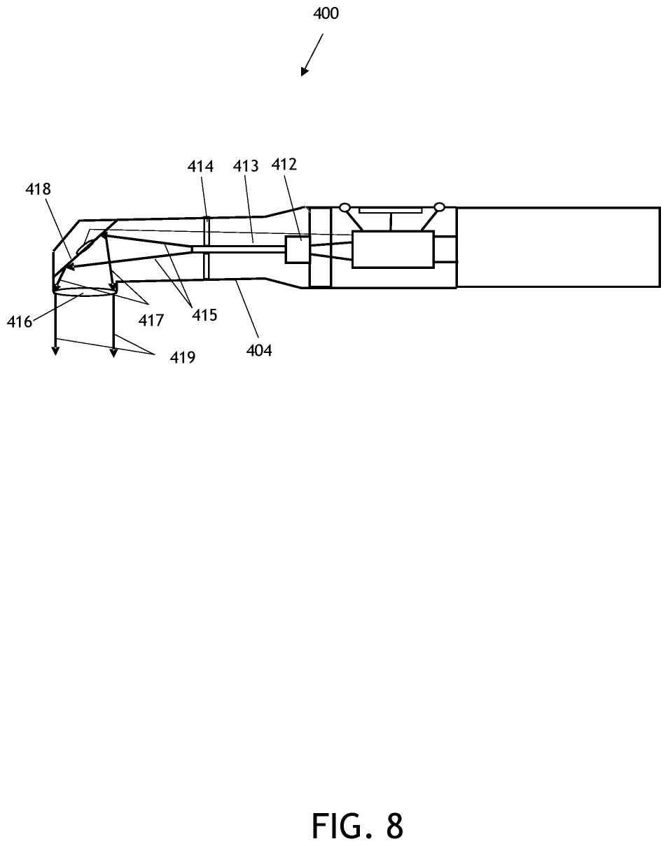

FIG. 8 depicts the same system as in FIG. 7, utilizing an alternate curing light head design (400). After laser module (412) emits a laser beam (415) through fiber (413), the beam (415) travels to a reflector (418) which directs beam (417) towards lens (416) positioned proximate the exit. Collimating lens (416) converts beam (417) into parallel beam (419) for use in curing applications. All the components after fiber holder (414) are in the head housing (404) and can be removed along with the head from the handpiece. The optical system which converts the light emitted from laser module (412) parallel beam (419) includes fiber (413), reflector (418), and collimating lens (416). In this embodiment, the collimated beam can be adjusted from 2 to 14 mm and the divergent angle of the beam θₐ is less than 10 degrees, however, additional variation in adjustment and the divergent angle are possible. The beam size can also be adjusted by attaching a fixture with different aperture size at beam exit point. The aperture size can be 2, 4, 6, and 8 mm. The fixture can also contain a filter to adjust beam intensity as needed. The length of the fiber (313) depends on the requirement of head (304) and can be ranged from 1 mm to 1000 mm. The laser module 312 can be placed in front of control circuit 306 or any position in or top or bottom of the circuit 306 or behind the circuit 306.

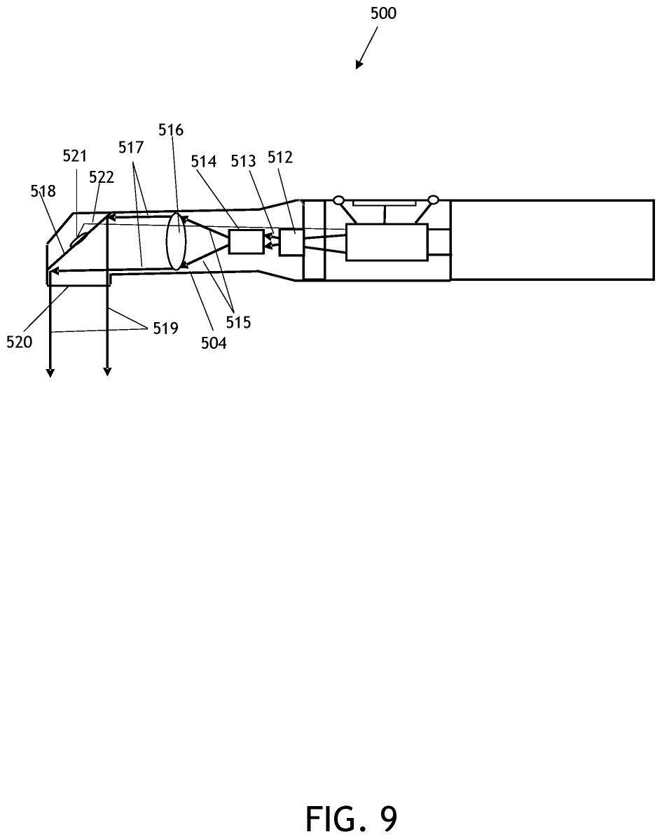

FIG. 9 also depicts the same system as in FIG. 7, utilizing an alternate curing light head design (500). In this embodiment laser module (512) emits a beam (513) into lens (514). It is common that the beam (513) to be an oval shape. Lens (514) may then focus the beam (513) to a point, then into a circular beam (515). There is a collimating lens (516) that converts the light beam (515) to a parallel beam (517). The position of the lenses relative to each other and laser module (312) will depend on their focal lengths and the size of the parallel beam will depend on diameter of the lens (516). It is possible for lenses (514) and (516) to be a single lens, depending on a design which will achieve a parallel beam from the emitted light directly from laser module (512). The parallel beam (517) travels to a reflector (518) which directs a reflected beam (519) to the wand exit (520). The components after laser module (512) will be in the head housing (504) and can be removed from the handpiece. The optical system which converts the light emitted from laser module (512) parallel beam (519) includes lenses (514) and (516), and reflector (518). In this embodiment, the collimated beam can be adjusted from 2 to 14 mm and the divergent angle of the beam θₐ is less than 10 degrees, however, additional variation in adjustment and the divergent angle are possible. The beam size can also be adjusted by attaching a fixture with different aperture size at beam exit point. The aperture size can be 2, 4, 6, and 8 mm. The fixture can also contain a filter to adjust beam intensity as needed.

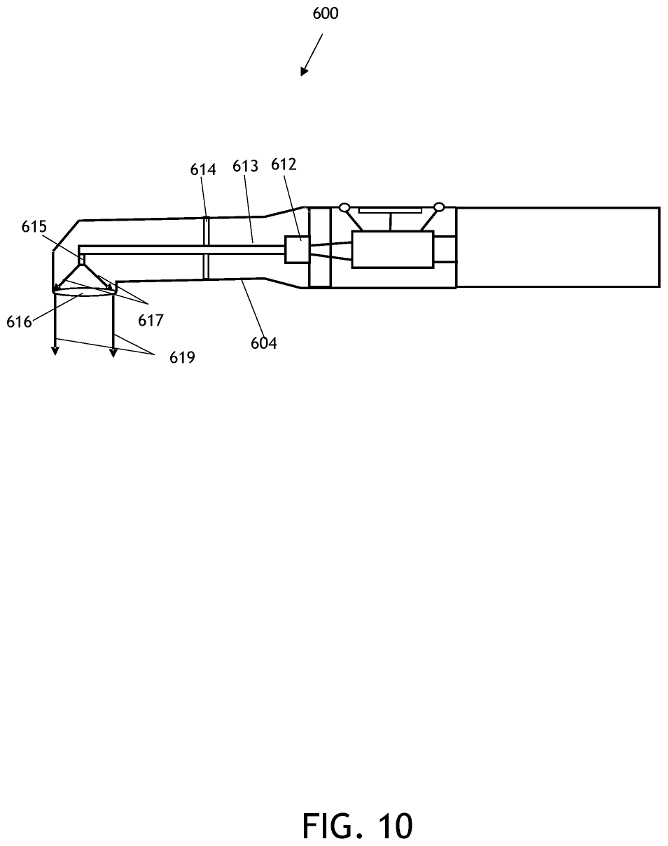

FIG. 10 also depicts the same system as in FIG. 7, utilizing another alternate curing light head design (600). In this embodiment, a fiber (613) may be attached to the laser module (612) and extends towards the end of head (604). It then makes a 90-degree turn (615) and points towards the light exit. A light beam emitted from the laser module (612) travels the fiber (613) and is emitted as beam (617). A collimating lens (616) situated at exit will convert the laser beam to a parallel beam (619). The direction of light exit in respect to horizontal axis of curing head is determined by the angle of fiber (615). The components after fiber holder (614) will be in the housing and can be removed from the handpiece while a holder (614) is provided to stabilize the length of fiber (613) extending from the laser module (612). The optical system which converts the light emitted from laser module (612) parallel beam (619) includes fiber components (613) and (615) and collimating lens (616). This is the same embodiment illustrated in FIG. 8, where the collimated beam can be adjusted in size from 2 to 14 mm and the divergent angle of the beam θₐ is less than 10 degrees. The length of the fiber (313) depends on the requirement of head (304) and can be ranged from 1 mm to 1000 mm. The laser module 312 can be placed in front of control circuit 306 or any position in or top or bottom of the circuit 306 or behind the circuit 306. The beam size can also be adjusted by attaching a fixture with different aperture size at beam exit point. The aperture size can be 2, 4, 6, and 8 mm. The fixture can also contain a filter to adjust beam intensity as needed.

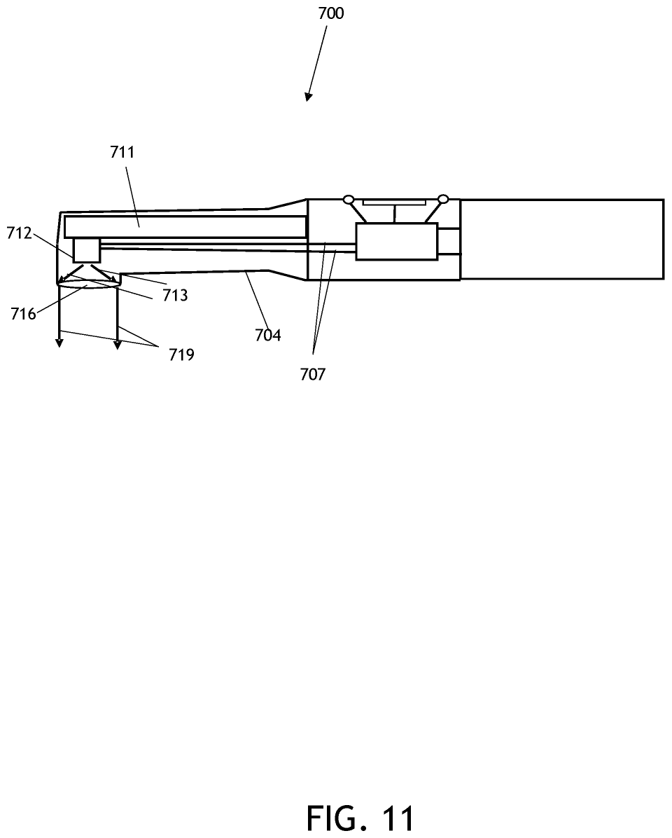

FIG. 11 also depicts the same system as in FIG. 7, utilizing another alternate curing light head design (700). In this embodiment, a heat sink (711) is positioned inside the head housing (704) along most of its length. Module connections (707) likewise extend into the head housing (704). A laser module (712) is attached to the heat sink (711) and connections (707) and emits a beam (713). The beam travels to a collimating lens (716) which is at an exit of head housing. The lens (716) converts the laser beam to a parallel beam (719). The direction of light exit in respect to horizontal axis of curing head is determined by the position of laser module (712) and lens (716). The laser module (712), its connection, and lens (716) are part of housing (704) and can be removed from handpiece if needed. The optical system which converts the light emitted from laser module (712) parallel beam (719) includes collimating lens (716). It is the same as the embodiment illustrated in FIG. 8, where the collimated beam can be adjusted in size from 2 to 14 mm and the divergent angle of the beam θₐ is less than 10 degrees. The beam size can also be adjusted by attaching a fixture with different aperture size at beam exit point. The aperture size can be 2, 4, 6, and 8 mm. The fixture can also contain a filter to adjust beam intensity as needed.

FIG. 12 also depicts the same system as in FIG. 7, utilizing a therapeutic laser head for use in surgery or other therapeutic applications (800). Laser module (812) is mounted upon heat sink (811) and has a fiber (813) attached thereto. The fiber (813) may be terminated with ferrule or be free standing with a cleaved interface on fiber side and the size or diameter of the fiber can be ranged from 50 to 1000 μm. A holder (814) secures the fiber (813) into a position and a coupler (815) is provided to align fiber (813) from laser module (812) to fiber (816) in the head housing (804). The head fiber (816) can also be terminated with a ferrule or be free standing with a cleaved interface. The coupler (815) shall have a tight tolerance to align the two fibers and ensure the laser beam transmitted from fiber (813) to head fiber (816) has a minimal loss. Coupler (815) may feature an optional lens (817) between fiber (813) and head fiber (816). The lens (817) can couple the light between fibers to increase transmission efficiency. The head fiber (816) is further extended to outside of housing (804) through a bendable tubing (818) which may be used to bend head fiber (816) to any angle as desired by bending the tube (818). All the components after fiber holder (814) in the head housing (804) can be removed along with the head from the handpiece. The length of the fiber (813) depends on the requirement of head (304) and can be ranged from 1 mm to 1000 mm. The laser module 312 can be placed in front of control circuit 306 or any position in or top or bottom of the circuit 306 or behind the circuit 306.

FIG. 13 also depicts the same system as in FIG. 7, utilizing a therapeutic laser head for use in surgery or other therapeutic applications (900). Some therapeutic applications only require a broad beam of light of a given frequency, this configuration (900) emits broad, non-cutting, non-collimated light. Laser module (912) is mounted upon heat sink (911) and has an attached fiber (913) extending therefrom. The fiber (913) may be terminated with ferrule or be free standing with a cleaved interface on fiber side. As in other embodiments, the size or diameter of the fiber can be ranged from 50 to 1000 μm. A holder (914) secures the fiber into a position. The fiber (913) provides a laser beam (915) in the housing (904) to a lens (916) which will enlarge and shape the beam with desired size and shape and guide the light (919) to conical exit (918). All the components after fiber holder (914) are in the head housing (904) and can be removed along with the head from the handpiece.

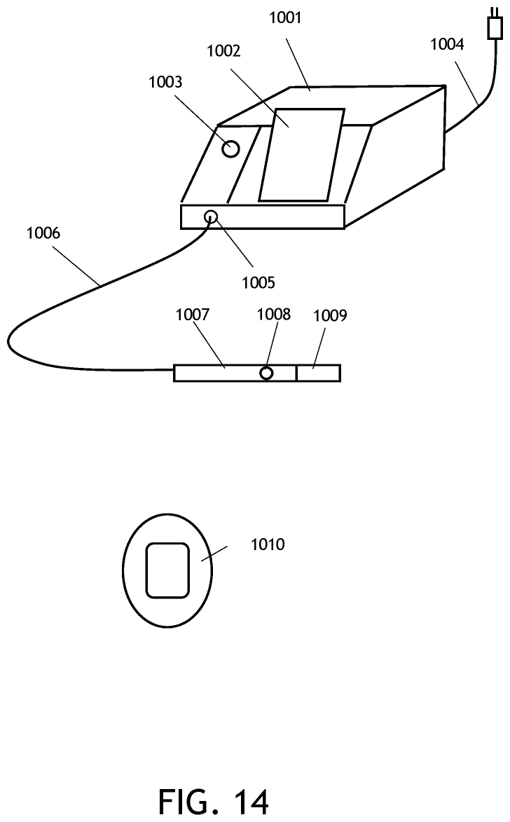

Any of the above heads may be utilized in a conventional operation unit, such as the desk top unit (1000) shown in FIG. 14. Desk top unit (1000) has a main body (1001) and a control display (1002) for system operations. The control display (1002) can be a touch pad, a touch screen, or removable module like iPad. Power is provided through power supply (1004) and the unit should have an emergency stop (1003). The system can utilize a rechargeable battery or AC/DC power input. Fiber (1005) and a control cable (1006) extend to a handpiece (1007) on which an attachment (1009) is attached. This handpiece attachment (1009) can be curing light tip, a fiber tip or a therapeutic tip as described above. Control switches may be located on the handpiece (1008) or through a footswitch, such as wireless footswitch (1010). The system can be controlled by either switch on hand piece (1008) or wireless foot switch (1010) while finer details may be controlled on the control display (1002). The beam size and attachment fixture are the same as other embodiments described in this article.

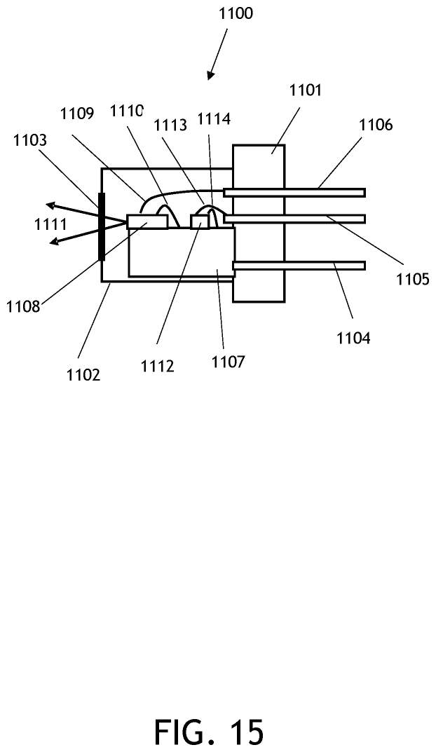

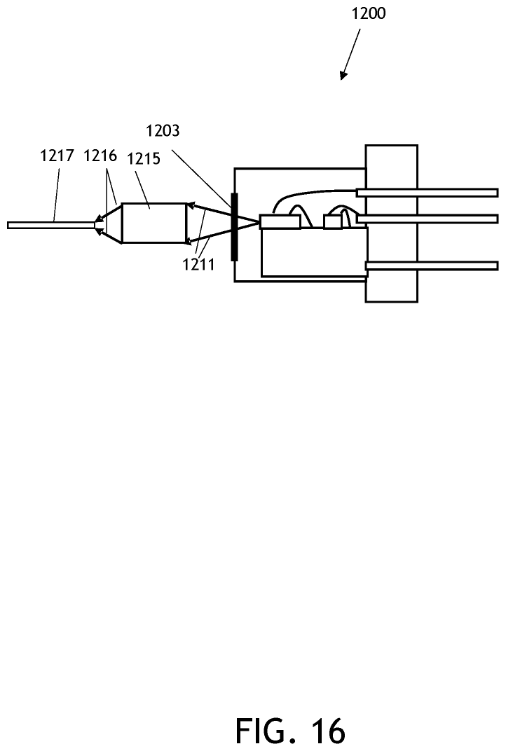

FIGS. 15 and 16 depict alternate embodiments for a laser module. In FIG. 15, laser module 1100 presents a base (1101) and casing (1102), where window (1103) in the casing (1102) allows emitted light to exit. The casing (1102) and base (1101) are generally made of metal or any heat conductive materials. There are electrodes for power input to the laser (1106) and detector (1105) chips inside laser module, where (1104) is a common electrode. Inside the casing, there is a heat sink (1107) attached to the base (1101). At least one laser chip (1108) is attached to a heat sink (1107) and to common electrode (1104) and chip electrode (1106) through wires (1109) and (1110) respectively. The laser chip (1108) shall emit the light required for system operations. The laser chip (1108) can be a single chip or a chip array or multiple chips and be made of AlGaInN, GaInP, AlGaAs, or other compounds. The wavelength of light emitted by laser chip (1108) shall be that or those required by the system. For example, wavelengths 400 nm-480 nm can be used for bacterial reduction and curing. Wavelengths ranged around 650 nm can be used for pain therapy. Typical wavelengths for curing composites or adhesives can be in the range from 280 nm to 520 nm. Typical wavelengths for surgical and other therapeutic uses can be 650 nm, 780 nm, 810 nm, 980 nm, 1064 nm, 1160 nm, 1320 nm, 1505 nm, and others. The laser module should be capable of emitting radiant energy at more than one discrete wavelength (about 50 nm apart). The beam emitting side of laser chip is aligned with window (803) and usually emits a divergent beam (1111). An optional photo detector (1112) may be attached to the heat sink (1107) or at any position inside the casing (1102). The photo detector (1112) may be used to monitor the laser chip emitting power as feedback for controlling laser output. The photo detector (1112) can be connected to detector electrode (1105) and common electrode (1104) through wires (1113) and (1114) respectively. The purpose of the photo detector (1112) is to measure the light from laser module (1100) and provide a feedback signal to the control circuit to control light beam emission of laser chip. An alternate laser module (1200) depicted in FIG. 16, features an optional lens structure proximate exit window (1203). Lens (1215) collects laser beam and converts it into a beam (1216) which will be focused to a point at end a fiber (1217). Thus, laser light is incorporated into a fiber for further transportation in the fiber. The diameter if the fiber can be ranged from 50 to 1000 μm and can be terminated with or without a ferrule. If multiple chips are used in the laser module, then an optical system needs to be utilized to incorporate the laser beams from multiple chips into the fiber.

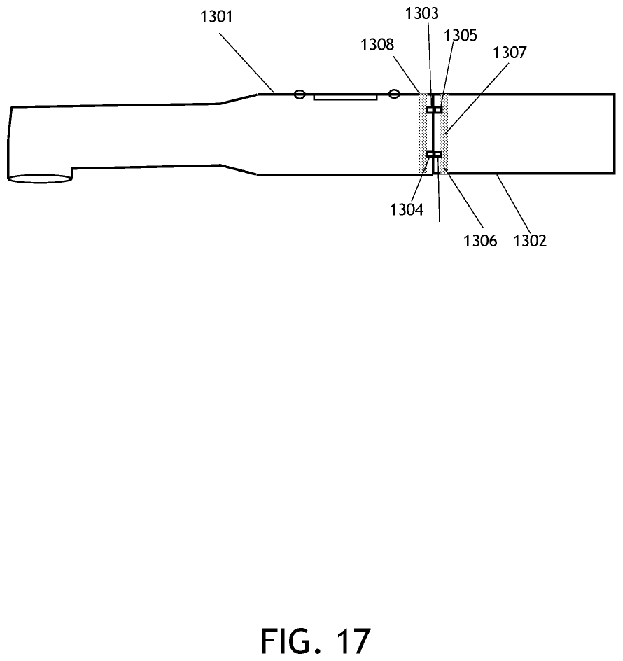

FIG. 17 illustrates one embodiment of battery attachment, which can be used as main power switch and emergency button for the laser units. Battery (1302) is attached to the handpiece of the light unit (1301). At end of handpiece (1301) are two electrical contacts (1303) and (1304). Battery (1302) also features two electrical contacts (1305) and (1306). The contacts in both bodies are mechanically aligned and contact each other when two bodies are attached. The attachment of two bodies may be facilitated using magnets, where at least one magnet (1307) is positioned in battery body and at least one other (1308) is positioned in the handpiece. In the practice, there may be magnets in either handpiece (1301) or battery body (1302) and the casing of the other is made of magnetically attractive materials like iron. The strength of magnets shall be selected to hold battery to main body when two bodies together and can also easily detached from handpiece.

Magnetic securement may also be utilized in securing the head to the handpiece as well. FIG. 14 illustrates one embodiment of head attachment for invented system where the handpiece of the curing light (1401) and the head (1402) are removable. At the end of handpiece (1401) there is at least one electrical contact (1403). In the head body (1402), there corresponding electrical contacts (1404). The contacts in both bodies are mechanically aligned and contact each other when two bodies are attached. The contact in each body can be multiple pins to transfer different signals. The bodies may be secured with magnets, where at least one magnet (1405) is positioned in curing head body (1402) and at least one other (1406) is positioned in handpiece body (1401). As with the battery described above, there may be one magnet or set of magnets in either handpiece or the curing head body and the casing of the other may contain magnetically attractive materials like iron. The strength of magnets shall be selected to hold curing head to main body when two bodies together and can also easily detached from the handpiece.

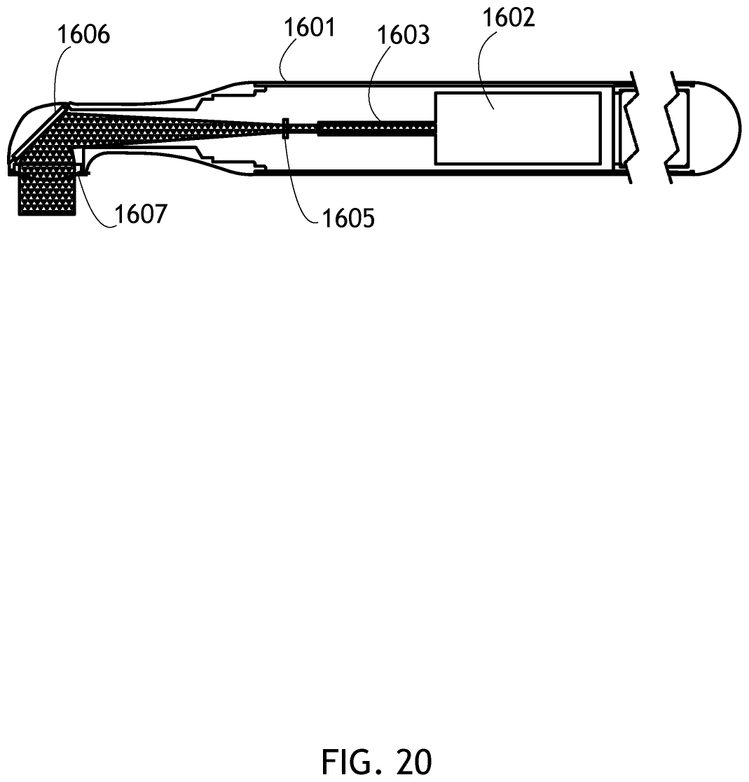

While a fiber with a divergent beam may be suitable for creating a collimated curing light, it must be remembered that the primary embodiment of the invention is to function as both a curing light and a laser. To this end, it may not be efficient to have a beam that is inherently too divergent. This dichotomy may be addressed by having a curing light head with a divergent lens (FIG. 20), where a lens may be positioned within the path of the beam to separate and diverge the beam before it is recollimated for curing. FIG. 20 depicts an exemplary tool with a generic housing (1601) that also functions as the handle that is designed to fit comfortably in an average size adult hand and at the same time is the house for all the components required to manufacture a laser curing light. Fiber-coupled laser diode (1602) produces laser light where it is emitted directly into a fiber optic cable (1603) that generally comprises a glass fiber optic core and an outer plastic cladding, which cladding is designed to protect the glass fiber. The laser source (1602) in this case can be the depicted fiber-coupled system or a direct emission laser diode because the use of an intermediate diverging lens (1605) allows both types to be utilized. The light emitted in this case can be nearly parallel and may be considered non-divergent. Thereafter the parallel light is passed through divergent lens (1605) wherein the laser light is diverged at a pre-determined angle, and then directed to the reflector (1606) where it is reflected into collimating lens (1607) where the light is aligned in the same direction. Once collimated, the laser light is then directed to a resin, a composite filling, or other dental material. It is the same as illustrated in FIG. 8, In this embodiment, the collimated beam can be adjusted from 2 to 14 mm and the divergent angle of the beam θₐ is less than 10 degrees. The length of the fiber (313) depends on the requirement of head (304) and can be ranged from 1 mm to 1000 mm. The laser module 312 can be placed in front of control circuit 306 or any position in or top or bottom of the circuit 306 or behind the circuit 306. An attachment fixture at the exit of light can be utilized to adjust beam size and intensity as other embodiments.

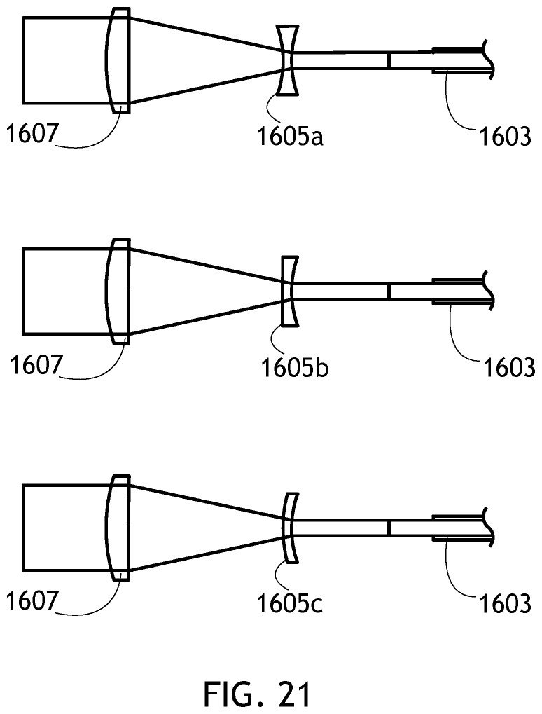

FIG. 21 depicts three different types of lenses which may be utilized to diverge the beam. In general, any lens which presents a concave surface towards the beam will diverge, such as the concave lens (1605a), the plano-concave lens (1605b), or the convex-concave complex lens (1605c). Each lens will then emit a divergent beam for collimation at the collimation lens (1607). Once the design and engineering principles of the present invention are understood it provides the means for those in the art to produce a custom laser curing light utilizing a compact laser source to then diverge the light into the correct beam dimensions and intensity to adequately cover a tooth or teeth and in less than 10 seconds cure a resin, composite, or other dental material.

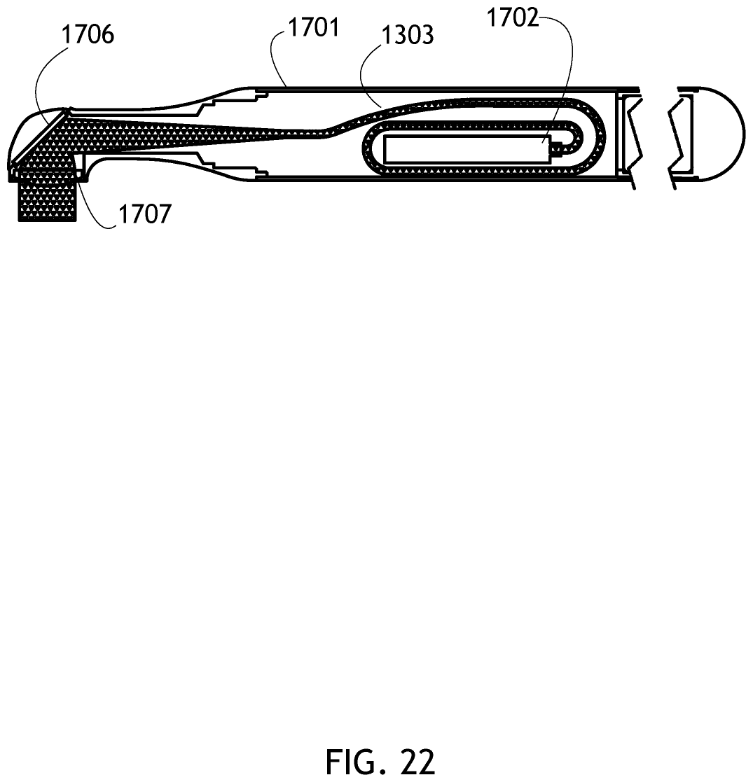

A further embodiment of invention may incorporate laser light diffusion methods to help uniformly blend the light into a more diffuse beam such that “dead spots” are reduced or entirely erased. Shorter lengths of fiber are more prone to have imperfections which may create areas within a beam where light does not shine. Long lengths of spooled fiber optic cable may be used to diffuse the light into a more uniform beam because longer lengths of fiber optic cable allow the light to diffuse as it bounces within the cable, especially a fiber optic cable longer than 0.5 inches (1.25 cm). To address this problem and utilize a longer length cable, the fiber cable is spooled or attached to the housing with as many turns as possible such that the longest length that can be achieved is designed into the housing itself. The purpose of the spooling is to increase the overall length the laser light must travel to diffuse the light in order to reduce any dead spots the light source produces. The incidents of corners and turns in the fiber optic cable will also help diffuse the dead spots. The present invention contemplates the spool to contain possibly 0.4-20 inches (10-51 cm) of fiber optic cable as shown in FIG. 22. In the depicted embodiment, a generic housing (1701) functions as the handle that is designed to fit comfortably in an average size adult hand and at the same time is the house for all the components required to manufacture a laser curing light. Fiber-coupled laser diode (1702) produces laser light where it is emitted directly into a fiber optic cable (1703) that generally comprises a glass fiber optic core and an outer plastic cladding, which cladding is designed to protect the glass fiber. The fiber optic cable (1703) is spooled within the housing (1701) as loosely as possible in order to place the least amount of stress on the fiber cable.

Although the present invention has been described with reference to preferred embodiments, numerous modifications and variations can be made and still the result will come within the scope of the invention. No limitation with respect to the specific embodiments disclosed herein is intended or should be inferred.

1. A system combining therapeutic lasers and curing lights, the system comprising:
a powered radiant emission source and a collection fiber operably in communication therewith to channel radiant energy from said radiant emission source and direct the radiant energy towards an emitter head mounted upon a handpiece; and
a plurality of exchangeable emitter heads, each emitter head selectable to a given purpose and said plurality of emitter heads including at least two emitter heads selected from the set of emitter heads consisting of:
a head that receives radiant energy from the collection fiber and directs said radiant energy into an energy divergence system, followed by a collimating lens, which then collimates the radiant energy and emits it as a curing light; and,
a head that receives radiant energy from the collection fiber and directs said radiant energy into a therapeutic fiber, which then concentrates the radiant energy into a focused, cutting laser;
wherein one emitter head is selected to transform and emit radiant energy according to a given procedure and attached to the system for use.

2. The system of claim 1, the radiant emission source being a laser module capable of emitting radiant energy at a plurality of discrete frequencies and the system further comprises a control system to select one discrete frequency to emit at a time.

3. The system of claim 1, the energy divergence system further comprising at least one energy transmittal fiber optic with a divergent numerical aperture.

4. The system of claim 1, the energy divergence system further comprising at least one divergent lens.

5. The system of claim 1, the system further comprising a coiled fiber optic resident within the handpiece.

6. The system of claim 1, each emitter head in the set of emitter heads being selectively magnetically secured to the handpiece.
